(19) United States
(12) Patent Application Publication
(10) Pub. No.: US 2010/0016637 A1
(43) Pub. Date: Jan. 21, 2010

(54) Method for Producing Oxime

(75) Inventors: Miyuki OIKAWA, Ichihara-Shi (JP); Masaru KITAMURA, Niihama-Shi (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: SUMITOMO CHEMICAL COMPANY, LIMITED, Tokyo (JP)

(21) Appl. No.: 12/501,779

(22) Filed: Jul. 13, 2009

(30) Foreign Application Priority Data
Jul. 15, 2008 (JP) 2008-183467

(51) Int. Cl. C07C 249/04

(52) U.S. Cl. 564/253

(57) ABSTRACT

The present invention provides a method for producing an oxime by carrying out an ammoximation reaction of a ketone with an organic peroxide and ammonia in a solvent in the presence of a titanosilicate, characterized in that the reaction is carried out by feeding the ketone and ammonia to a reactor in which the solvent, the titanosilicate and the organic peroxide are charged.

### FIELD OF THE INVENTION

The present invention relates to a method for producing an oxime by ammoximation reaction of a ketone. The oxime is useful as a starting material of amide or lactam.

### BACKGROUND OF THE INVENTION

Ammoximation reaction of a ketone with hydrogen peroxide and ammonia in a solvent in the presence of a titanosilicate has been known in the art (JP 2006-169168 A, JP 2007-1952 A and JP 2007-238541 A). However, hydrogen peroxide has not been always cost-effective. Hence, a method of using an organic peroxide as a recyclable peroxide has been studied but is not always satisfactory in that the selectivity or yield of the oxime is not stable for industrial production.

### SUMMARY OF THE INVENTION

The present invention provides a method for producing an oxime, which comprises reacting ketone with an organic peroxide, and ammonia in a solvent in the presence of titanosilicate, wherein the ketone and ammonia are fed into a reactor containing the solvent, titanosilicate and the organic peroxide.

According to the present invention, an oxime can be produced with stable selectivity and yield.

### DESCRIPTION OF THE PREFERRED EMBODIMENTS

The ketone that may be used in the present invention is typically an aliphatic ketone, an alicyclic ketone, unsaturated ketone, or an aromatic ketone. The ketones may be used alone or as a mixture of two or more of them. Examples of the aliphatic ketone include dialkyl ketones such as acetone, ethyl methyl ketone and isobutyl methyl ketone. Examples of the unsaturated ketone include alkyl alkenyl ketones such as mesityl oxide. Examples of the aromatic ketone include alkyl aryl ketones such as acetophenone; diaryl ketones such as benzophenone. Examples of the alicyclic ketone include cycloalkanones such as cyclopentanone, cyclohexanone, cyclooctanone and cyclododecanone. Examples of the unsaturated ketone also include cycloalkenones such as cyclopentenone and cyclohexenone. Among these ketones, cycloalkanones are preferably used in the present invention.

The ketone may be obtained by oxidation of an alkane, oxidation (dehydrogenation) of a secondary alcohol, or hydration and oxidation (dehydrogenation) of an alkene.

Ammonia may be used in the form of a gas, liquid or solution in an organic solvent. The amount of ammonia is preferably adjusted so that the concentration of ammonia in the liquid phase of the reaction mixture becomes 1% by weight or more. By adjusting the concentration of ammonia in the liquid phase of the reaction mixture to the predetermined value or more, the conversion rate of a ketone and the selectivity of an oxime can be increased, and thus the yield of an oxime as the objective product can also be increased. The concentration of ammonia is preferably 1.5% by weight or more, and usually 10% by weigh or less, preferably 5% by weight or less. The amount of ammonia is usually 1 mol or more, and preferably 1.5 mol or more, per 1 mol of the ketone.

The solvent that may be used for the ammoximation reaction is preferably a water-soluble organic solvent, more preferably nitriles such as acetonitrile, propionitrile, butyronitrile, isobutyronitrile, trimethylacetonitrile, valeronitrile, isovaleronitrile and benzonitrile; and alcohols such as methyl alcohol, ethyl alcohol, n-propyl alcohol, isopropyl alcohol, n-butyl alcohol, s-butyl alcohol, t-butyl alcohol and t-amyl alcohol, and still more preferably a nitrile or alcohol having up to 2 carbon atoms. If necessary, two or more kinds of them can be used. In the present invention, the water content in the liquid phase of the reaction mixture is preferably kept lower in view of the selectivity of the oxime.

The amount of the solvent is usually from 1 to 500 parts by weight, and preferably from 2 to 300 parts by weight, per 1 part by weight of the ketone.

The titanosilicate that may be used in the present invention typically is a titanosilicate that contains titanium, silicon and oxygen as elements constituting its framework, and the framework may be composed of titanium, silicon and oxygen. Alternatively, the titanosilicate may contain elements other than titanium, silicon and oxygen, for example, boron, aluminum, gallium, iron, and chromium as elements constituting the framework. Specific examples of the titanosilicate include Ti-MWW as a crystalline titanosilicate having an MWW structure, TS-1 as a crystalline titanosilicate having an MFI structure, Ti-MCM-41 as a noncrystalline titanosilicate having a mesopore structure and the like. “MWW” and “MFI” are structural codes of zeolite defined by the International Zeolite Association (IZA).

The amount of the titanosilicate is usually adjusted within a range from 0.1 to 10% by weight relative to the liquid phase of the reaction mixture.

Examples of the organic peroxide that may be used include organic hydroperoxides such as t-butyl hydroperoxide, cumene hydroperoxide, cyclohexyl hydroperoxide, diisopropylbenzene hydroperoxide, p-methane hydroperoxide and 1,1,3,3-tetramethylbutyl hydroperoxide; dialkyl peroxides such as t-butylcumyl peroxide, di-t-butyl peroxide, di-t-hexyl peroxide, dicumyl peroxide, α,α′-di(t-butylperoxy)diisopropylbenzene, 2,5-dimethyl-2,5-di(t-butylperoxy)hexane and 2,5-dimethyl-2,5-bis(t-butylperoxy)hexyne-3; peroxy esters such as cumyl peroxy neodecanoate, 1,1,3,3-tetramethylbutyl peroxy neodecanoate, t-hexyl peroxy neodecanoate, t-butyl peroxy neodecanoate, t-butyl peroxy neoheptanoate, t-hexyl peroxy valeate, t-butyl peroxy pivalate, 2,5-dimethyl-2,5-di(2-ethylhexanoylperoxy)hexane, 1,1,3,3-tetramethylbutyl peroxy-2-ethyl hexanoate, t-hexyl peroxy-2-ethyl hexanoate, t-butyl peroxy-2-ethyl hexanoate, t-butyl peroxy laurate, t-butyl peroxy-3,5,5-trimethyl hexanoate, t-hexyl peroxy isopropyl monocarbonate, t-butyl peroxy-2-ethylhexyl monocarbonate, 2,5-dimethyl-2,5-di(benzoylperoxy)hexane, t-butyl peroxy acetate, t-hexyl peroxy benzoate and t-butyl peroxy benzoate; diacyl peroxides such as disiobutyryl peroxide, di(3,5,5-trimethylhexanoyl)peroxide, dilauroyl peroxide, disuccinic acid peroxide, dibenzoyl peroxide and di(4-methylbenzoyl)peroxide; and peroxy dicarbonates such as diisopropyl peroxy dicarbonate, di-n-propyl peroxy dicarbonate, bis(4-t-butylcyclohexyl)peroxy dicarbonate, di-2-ethylhexyl peroxy dicarbonate and di-sec-butyl peroxy dicarbonate. Among these organic peroxides, hydroperoxides are preferred.

In the ammoxidation reaction of the present invention, the organic peroxides are converted to an alcohol and/or carboxylic acid, which can be collected by distillation, extraction or the like for reuse as the organic peroxide, hence the present process is desirable for saving cost.

The amount of the organic peroxide that may be used is usually from 0.5 to 20 moles, and preferably from 0.5 to 10 moles, per 1 mol of a ketone.

Next, the mode of the ammoximation reaction will be explained. In the present invention, first, a solvent, a titanosilicate and an organic peroxide are introduced to a reactor. There is no particular limitation with respect to the order of introduction of them. Then, ketone and ammonia are typically fed to the reactor in which the titanosilicate is suspended by stirring. The ketone and ammonia are typically fed simultaneously as a ketone feed and an ammonia feed, which is referred to as “co-feed” in this specification, or as a mixture thereof. Preferably, a portion or ammonia may be preliminarily charged or fed together with the organic peroxide, and then the ketone and the remaining ammonia may be fed to the reactor. Alternatively, the organic peroxide may be preliminarily fed to the reactor and then the ketone, ammonia, and additional organic peroxide may be fed together.

The ammoximation reaction may be carried out batchwise or continuously. Particularly preferred is a continuous reaction process which comprises withdrawing a liquid phase of the resulting reaction mixture containing the product, and feeding reactants, typically ketone and ammonia, simultaneously in view of productivity and operability.

For example, a continuous reaction is preferably carried out by preparing a reaction mixture in which titanosilicate is suspended in a reactor, feeding reaction starting materials such as ketone and ammonia to the reactor, and withdrawing a liquid phase of the reaction mixture from the reactor through a filter.

Preferably employed is a reactor lined with glass or made of stainless steel for preventing of decomposition of the organic peroxide.

The temperature of the ammoximation reaction is usually from 50 to 200° C., and preferably from 80 to 150° C. The reaction pressure may be normal pressure, and is usually from 0.2 to 1 MPa absolute pressure, and preferably from 0.2 to 0.5 MPa, so as to readily dissolve ammonia in the liquid phase of the reaction mixture. The reaction pressure may be adjusted by using an inert gas such as nitrogen or helium.

The post-treatment operation of the resulting reaction mixture is appropriately selected. For example, an oxime can be separated by separating a titanosilicate from the reaction mixture through filtration or decantation and distillating a liquid phase.

### EXAMPLES

The present invention will be explained by way of the following examples and comparative examples, but it is not construed to limit the present invention thereto. In the following examples, the liquid phase of the reaction mixture was analyzed by gas chromatography, and the conversion rate of cyclohexane as well as the selectivity and yield of cyclohexanone oxime were calculated based on the results of the analysis.

### Example 1

In a 1 L autoclave (reactor), 158.7 g of an acetonitrile solution containing 2.6% by weight of ammonia, 7.6 g of a cumene solution containing 80% by weight of cumene hydroperoxide and 5.0 g of Ti-MWW (prepared by the same manner as described in Chemistry Letters, 2000, pp. 774-775) were charged and a vapor phase portion in the reactor was replaced by nitrogen. After the reactor was sealed, the temperature in the reactor was raised to 110° C. under stirring. The pressure in the reactor was 0.5 MPa. Next, 10 g of an acetonitrile solution containing 4.7% by weight of cyclohexanone, and 115 g of an acetonitrile solution containing 3.9% by weight of ammonia each were fed (co-fed) to the reactor over 1 hour. The concentration of ammonia in the liquid phase of the reaction mixture changed within a range from 1.0 to 2.6% by weight relative to the liquid phase.

After the co-feeding, the liquid phase of the reaction mixture was withdrawn and analyzed by gas chromatography. The conversion rate of cyclohexanone was found 73.3%, the selectivity to cyclohexanone oxime was 71.2% and the yield of cyclohexanone oxime was 52.2%. Selectivity to cyclohexanoneimine (a compound produced by imination of cyclohexanone) and impurities derived from the imine, based on the consumed cyclohexanone, was 21.1%.

### Example 2

In a 1 L autoclave (reactor), 158.7 g of an acetonitrile solution containing 2.6% by weight of ammonia, 5.5 g of an n-decane solution containing 65% by weight of t-butyl hydroperoxide and 5.0 g of Ti-MWW (prepared by the same manner as described in Chemistry Letters, 2000, pp. 774-775) were charged, and a vapor phase portion in the reactor was replaced by nitrogen. After the reactor was sealed, the temperature in the reactor was raised to 110° C. under stirring. The pressure in the reactor was 0.5 MPa. Next, 10 g of an acetonitrile solution containing 4.7% by weight of cyclohexanone, and 115 g of an acetonitrile solution containing 3.8% by weight of-ammonia each were fed (co-fed) to the reactor over 1 hour. The concentration of ammonia in the liquid phase of the reaction mixture changed within a range from 1.0 to 2.6% by weight relative to the liquid phase.

After the co-feeding, the liquid phase of the reaction mixture was withdrawn and analyzed by gas chromatography. The conversion rate of cyclohexanone was found 99.9%, the selectivity to cyclohexanone oxime was 63.6% and the yield of cyclohexanone oxime was 63.5%. Selectivity to cyclohexanoneimine (a compound produced by imination of cyclohexanone) and impurities derived from the imine, based on the consumed cyclohexanone, was 36.4%.

### Example 3

In a 1 L autoclave (reactor), 158.8 g of an ethanol solution containing 4.3% by weight of ammonia, 7.6 g of a cumene solution containing 80% by weight of cumene hydroperoxide and 5.0 g of Ti-MWW (prepared by the same manner as described in Chemistry Letters, 2000, pp. 774-775) were charged and a vapor phase portion in the reactor was replaced by nitrogen. After the reactor was sealed, the temperature in the reactor was raised to 110° C. under stirring. The pressure in the reactor was 0.5 MPa. Next, 10 g of an ethanol solution containing 4.7% by weight of cyclohexanone, and 115 g of an ethanol solution containing 3.8% by weight of ammonia each were fed (co-fed) to the reactor over 1 hour. The concentration of ammonia in the liquid phase of the reaction mixture changed within a range from 2.5 to 4.3% by weight relative to the liquid phase.

After the co-feeding, the liquid phase of the reaction mixture was withdrawn and analyzed by gas chromatography. The conversion rate of cyclohexanone was found 96.6%, the selectivity to cyclohexanone oxime was 37.3% and the yield of cyclohexanone oxime was 36.1%. Selectivity to cyclohexanoneimine (a compound produced by imination of cyclohexanone) and impurities derived from the imine, based on the consumed cyclohexanone, was 55.8%.

### Example 4

In a 1 L autoclave (reactor), 159.4 g of an acetonitrile solution containing 2.3% by weight of ammonia, 7.6 g of a cumene solution containing 80% by weight of cumene hydroperoxide and 5.0 g of Ti-MWW (prepared by the same manner as described in Chemistry Letters, 2000, pp. 774-775) were charged and a vapor phase portion in the reactor was replaced by nitrogen. After the reactor was sealed, the temperature in the reactor was raised to 115° C. under stirring. The pressure in the reactor was 0.5 MPa. Next, 10 g of an acetonitrile solution containing 4.7% by weight of cyclohexanone, and 115 g of an acetonitrile solution containing 3.8% by weight of ammonia and 1.0% by weight of cumene hydroperoxide each were fed (co-fed) to the reactor over 1 hour. The concentration of ammonia in the liquid phase of the reaction mixture changed within a range from 1.0 to 2.3% by weight relative to the liquid phase.

After the co-feeding, the liquid phase of the reaction mixture was withdrawn and analyzed by gas chromatography. The conversion rate of cyclohexanone was found 99.5%, the selectivity to cyclohexanone oxime was 83.9% and the yield of cyclohexanone oxime was 83.5%. Selectivity to cyclohexanoneimine (a compound produced by imination of cyclohexanone) and impurities derived from imine, based on the consumed cyclohexanone, was 16.0%.

### Comparative Example 1

In a 1 L autoclave (reactor), 273 g of an acetonitrile solution containing 1.8% by weight of ammonia, 3.8 g of a cumene solution containing 80% by weight of cumene hydroperoxide, 5.0 g of Ti-MWW (prepared by the same manner as described in Chemistry Letters, 2000, pp. 774-775) and 2.0 g of cyclohexanone were charged and a vapor phase portion in the reactor was replaced by nitrogen. After the reactor was sealed, the temperature in the reactor was raised to 90° C. under stirring. The pressure in the reactor was 0.2 MPa.

Next, stirring was carried out at the same temperature under the same pressure for 2 hours. Next, the liquid phase of the reaction mixture was withdrawn and analyzed by gas chromatography. The conversion rate of cyclohexanone was found 37.9%, the selectivity to cyclohexanone oxime was 26.6% and the yield of cyclohexanone oxime was 10.1%. Selectivity to cyclohexanoneimine (a compound produced by imination of cyclohexanone) and impurities derived from the imine, based on the consumed cyclohexanone, was 18.1%.

### Comparative Example 2

In a 1 L autoclave (reactor), 256 g of an ethanol solution containing 2.1% by weight of ammonia, 3.9 g of a cumene solution containing 80% by weight of cumene hydroperoxide, 5.0 g of Ti-MWW (prepared by the same manner as described in Chemistry Letters, 2000, pp. 774-775) and 2.0 g of cyclohexanone were charged and a vapor phase portion in the reactor was replaced by nitrogen. After the reactor was sealed, the temperature in the reactor was raised to 90° C. under stirring. At this time, the pressure in the reactor was 0.2 MPa. Next, stirring was carried out at the same temperature under the same pressure for 2 hours. Next, the liquid phase of the reaction mixture was withdrawn and analyzed by gas chromatography. The conversion rate of cyclohexanone was found 41.1%, selectivity to cyclohexanone oxime was 8.3% and the yield of cyclohexanone oxime was 3.4%. Selectivity of the cyclohexanoneimine (a compound produced by imination of cyclohexanone) and impurities derived from the imine, based on the consumed cyclohexanone, was 25.7%.

### Comparative Example 3

In a 1 L autoclave (reactor), 243 g of a t-butanol solution containing 7.4% by weight of ammonia (also containing 12.5% by weight of water), 3.8 g of a cumene solution containing 80% by weight of cumene hydroperoxide, 10.0 g of TS-1 (prepared by the same manner as described in JP 56-96720 A) and 2.0 g of cyclohexanone were charged and a vapor phase portion in the reactor was replaced by nitrogen. After the reactor was sealed, the temperature in the reactor was raised to 90° C. under stirring. The pressure in the reactor was 0.2 MPa.

Next, stirring was carried out at the same temperature under the same pressure for 6 hours. Then, the liquid phase of the reaction mixture was withdrawn and analyzed by gas chromatography. The conversion rate of cyclohexanone was found 10.9%, the selectivity to cyclohexanone oxime was 3.2% and the yield of cyclohexanone oxime was 0.4%. Selectivity to cyclohexanoneimine (a compound produced by imination of cyclohexanone) and impurities derived from the imine, based on the consumed cyclohexanone, was 5.9%.

1. A method for producing an oxime, which comprises reacting a ketone with an organic peroxide and ammonia in a solvent in the presence of titanosilicate, wherein the ketone and ammonia are fed into a reactor containing the solvent, titanosilicate and the organic peroxide.

2. The method according to claim 1, wherein the ketone is a cycloalkanone.

3. The method according to claim 1, wherein the solvent is a water-soluble organic solvent.

4. The method according to claim 1, wherein the concentration of ammonia in a liquid phase of the resulting reaction mixture is adjusted to 1% by weight or more relative to the liquid phase.

5. The method according to claim 2, wherein the solvent is a water-soluble organic solvent.

6. The method according to claim 2, wherein the concentration of ammonia in a liquid phase of the resulting reaction mixture is adjusted to 1% by weight or more relative to the liquid phase.

7. The method according to claim 3, wherein the concentration of ammonia in a liquid phase of the resulting reaction mixture is adjusted to 1% by weight or more relative to the liquid phase.
